(19) United States
(12) Patent Application Publication
(10) Pub. No.: US 2024/0379200 A1
(43) Pub. Date: Nov. 14, 2024

(54) INFORMATION EXTRACTION WITH LARGE LANGUAGE MODELS

(71) Applicant: NEC Laboratories America, Inc., Princeton, NJ (US)

(72) Inventors: Xujiang Zhao, Hillsborough, NJ (US); Haifeng Chen, West Windsor, NJ (US); Wei Cheng, Princeton Junction, NJ (US); Yanchi Liu, Monmouth Junction, NJ (US); Zhengzhang Chen, Princeton Junction, NJ (US); Haoyu Wang, Plainsboro, NJ (US)

(21) Appl. No.: 18/649,145

(22) Filed: Apr. 29, 2024

Related U.S. Application Data
(60) Provisional application No. 63/500,664, filed on May 8, 2023.
(60) Provisional application No. 63/522,731, filed on Jun. 23, 2023.

(51) Int. Cl. G16H 10/60; G06F 40/40

(52) U.S. Cl. CPC G16H 10/60; G06F 40/40

(57) ABSTRACT

Methods and systems for information extraction include configuring a language model with an information extraction instruction prompt and at least one labeled example prompt. Configuration of the language model is validated using at least one validation prompt. Errors made by the language model in response to the at least one validation prompt are corrected using a correction prompt. Information extraction is performed on an unlabeled sentence using the language model to identify a relation from the unlabeled sentence. An action is performed responsive to the identified relation.

### RELATED APPLICATION INFORMATION

This application claims priority to U.S. patent application Ser. No. 63/500,664, filed on May 8, 2023, and to U.S. patent application Ser. No. 63/522,731, filed on Jun. 23, 2023,incorporated herein by reference in their entirety.

### BACKGROUND

### Technical Field

The present invention relates to information extraction and, more particularly, to the use of large language models to extract information from text.

### Description of the Related Art

Information extraction is a natural language task where unstructured text is analyzed to identify types of information that it contains. For example, information extraction may identify named entities within the input text and may further identify relationships between such named entities.

### SUMMARY

A method for information extraction includes configuring a language model with an information extraction instruction prompt and at least one labeled example prompt. Configuration of the language model is validated using at least one validation prompt. Errors made by the language model in response to the at least one validation prompt are corrected using a correction prompt. Information extraction is performed on an unlabeled sentence using the language model to identify a relation from the unlabeled sentence. An action is performed responsive to the identified relation.

A system for information extraction includes a hardware processor and a memory that stores a computer program. When executed by the hardware processor, the computer program causes the hardware processor to configure a language model with an information extraction instruction prompt and at least one labeled example prompt, to validate configuration of the language model using at least one validation prompt, to correct errors made by the language model in response to the at least one validation prompt using a correction prompt, to perform information extraction on an unlabeled sentence using the language model to identify a relation from the unlabeled sentence, and to perform an action responsive to the identified relation.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block/flow diagram of an information extraction task that is performed on a natural language input by a large language mode, in accordance with an embodiment of the present invention;

FIG. 2 is a block/flow diagram of an information extraction task that is performed on a natural language input by a large language mode, in accordance with an embodiment of the present invention;

FIG. 3 is a block/flow diagram of a series of prompts to a large language model that configure the large language model to perform domain-specific information extraction, in accordance with an embodiment of the present invention;

FIG. 4 is a block diagram showing the use of information extraction in the context of medical decision making in a healthcare facility, in accordance with an embodiment of the present invention;

FIG. 5 is a block diagram of a computing device that can configure and use a large language model to perform information extraction, in accordance with an embodiment of the present invention;

FIG. 6 is a block diagram showing an exemplary neural network architecture that can be used as part of the large language model, in accordance with an embodiment of the present invention; and

FIG. 7 is a block diagram showing an exemplary deep neural network architecture that can be used as part of the large language model, in accordance with an embodiment of the present invention.

### DETAILED DESCRIPTION OF PREFERRED EMBODIMENTS

Large language models can be used to perform a wide variety of natural language tasks, including information extraction. However, using a general purpose language model to perform a specific task can give rise to inaccuracies, as the language model is not specifically trained to perform the task. Similarly the language model may not have access to domain-specific information, reducing accuracy for domain-specific inputs. Furthermore, the construction of the prompt for the model has a large effect on the quality of the output.

To that end, specific-domain open information extraction using a large language model may be enhanced by in-context learning. Given an input sentence, the most important relations may be extracted, including a subject, an action, and an object. A set of domain-specific sentences with ground-truth labels and an associated confident label may be used, with the ground-truth labels including a subject, action, and object. An initial prompt may be designed for a general purpose language model, including a description of the information extraction task. A series of prompts may be provided some of with the domain-specific sentences and their associated labels to provide domain-specific knowledge to the language model. Additional prompts may be used to test the understanding of the language model and to provide corrections when the model generates erroneous results. The language model can then be used to extract information from unlabeled new sentences and to provide a confidence score as to the correctness of its output.

Referring now to FIG. 1, an exemplary information extraction task is shown. Input text 102 is provided to a large language model 104. The input sentences 102 are made part of one or more prompts, which instruct the large language model 104 to perform information extraction. In this example, the information extraction task specifically extracts subjects, actions, and objects and generates output 106, but it should be understood that any appropriate information extraction task may be performed instead. For example, some embodiments may further extract adverbs.

Any appropriate language model can be used for the large language model 104. Exemplary language models include ChatGPT, GPT3, and GPT4. The large language model may be implemented as a machine learning model, such as a transformer neural network model, having parameters that are pretrained on a large corpus of textual material.

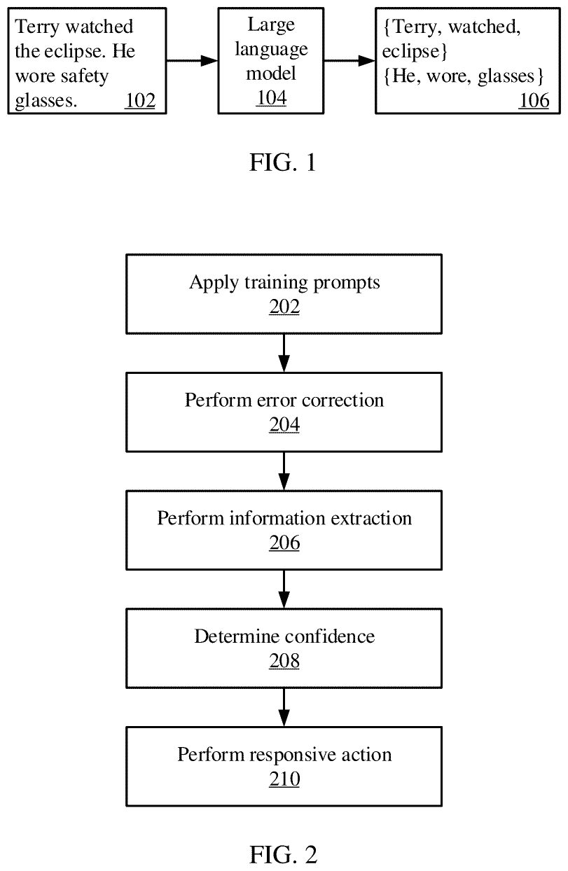

Referring now to FIG. 2, a method for performing information extraction with a large language model is shown. Block 202 applies training prompts to the language model 104, defining the information extraction task and providing examples from a set of labeled training data. The training data may pertain to a particular domain that was not used in the original training of the language model 104.

Block 204 may then perform error correction, for example by prompting the language model 104 to perform information extraction on additional examples from the labeled training data and comparing its predictions to the predetermined labels. The error correction of block 204 may include further prompts to the language model 104 instructing it as to the correct answer in instances when performs the task inaccurately.

Once the language model 104 has been properly instructed as to the task, block 206 performs information extraction on a new sentence input and block 208 determines a confidence associated with the extracted information. The confidence score may be provided by a human annotator, such as a subject-matter expert. Based on the output of the language model 104, block 210 may perform a responsive action. For example, this action may take place in the context of a medical treatment at a healthcare facility, as described in greater detail below. The responsive action may be made conditional on a threshold level of confidence, such as at least 90%.

For a set of training examples x, there may be a set of sentences S={S₁, S₂, . . . , S_{N}}, where Nis the number of sentences in the set, and a set of respective ground truth labels Y={y₁, . . . , y_{N}}, where each yₙ={(sₙ¹, aₙ¹, oₙ¹), . . . , (sₙ^{K}, aₙ^{K}, oₙ^{K})}, where sₙ^{k} represents a subject, aₙ^{k} represents an action, oₙ^{k} represents an object, and K is the number of relations for the sentence Sₙ.

The training prompts include a definition of the role for the system. For example, this may be expressed in natural language as a command or instruction, such as, “You are an AI assistant for open information extraction.” Subsequent training prompts provide in-context learning, for example defining a format of the relations that are to be extracted. For example, such a training prompt may be expressed as:

“Identify all combinations of subject, action, and object for any given sentences, and present them in the form of triplets: (subject, action, object). Here are m examples:

Sentence: ‘domain specific sentence: S₁’

Relations:

1. (s₁¹, a₁¹, o₁¹)

2. (s₁², a₁², o₁²)

. . .

K. (s₁^{K}, a₁^{K}, o₁^{K})

Sentence: ‘domain specific sentence: S₂’

Relations:

1. (s₂¹, a₂¹, o₂¹)

2. (s₂², a₂², o₂²)

. . .

K. (s₂^{K}, a₂^{K}, o₂^{K})

Sentence: ‘domain specific sentence: Sₘ’

Relations:

1. (sₘ¹, aₘ¹, oₘ¹)

2. (sₘ², aₘ², oₘ²)

. . .

K. (sₘ^{K}, aₘ^{K}, oₘ^{K})

Now please identify all combinations of subject, action, and object for the m sentences.”

Based on this in-context learning prompt, the language model 104 learns domain-

specific knowledge and predicts open information for an input sentence. Thus for an input sentence p, the extracted information may be denoted herein as rₚ.

The error correction of block 204 helps the language model 104 adapt to the

domain of the training data. Given training examples {S, Y}, the language model 104 predicts open information contained therein. Given a sentence Sₙ, the language model prediction rₚ is a set of extracted relations. An error correction prompt may be generated, for example as:

“No, the correct combinations of relations are:

1. (sₘ¹, aₘ¹, o_{M1}¹)

2. (sₘ², aₘ², oₘ²)

. . .

K. (sₘ^{K}, aₘ^{K}, oₘ^{K})”

Based on the error correction prompt, the large language model is able to generate higher-accuracy predictions for information extraction, denoted here in as îₚ.

Blocks 202 and 204 may further provide instructions to the language model 104 regarding prediction of confidence scores. For example, a prompt may be constructed as:

“For this sentence p, identify all combinations of subject, action, and object for any given sentences, and also provide the confidence for each combination.”

This prompt generates a confidence score c along with the prediction rₚ. The confidence score may be defined as c=[c₁, . . . , Cₖ] and the ground truth confidence may be represented as ĉ. A further prompt may then be issued to perform error correction, for example as:

“No, your confidence is not correct. The correct confidence is ê.”

After the confidence correction, the language model 104 can provide its confidence prediction via a further prompt:

“For this sentence p, please provide the confidence of the relation rₚ.”

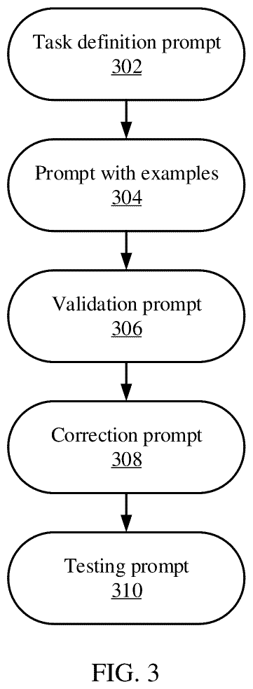

Referring now to FIG. 3, a sequence of prompts is shown. The first prompt is a task definition prompt 302, which instructs the language model 104 as to what type of information is being extracted. This is followed by a prompt with a set of examples drawn from the training data. These two prompts provide information to the language model 104 as to how to respond to subsequent prompts and further provide contextual information regarding any domain-specific knowledge that may be encoded in the examples.

The initial setup is followed by a validation prompt 306, which draws examples from the labeled training data and asks the primed language model 104 to predict the predetermined labels for those examples. For any examples where the language model 104 provides incorrect answers, a correction prompt 308 is used to identify what example the language model 104 predicted incorrectly and what the correct answers should have been.

After the correction has been performed, additional validation prompts 306 may be used to ensure the language model 104 is operating correctly. If so, a testing prompt 310 with a new sentence or sentence may be applied to the language model 104 to extract information and to generate a confidence score.

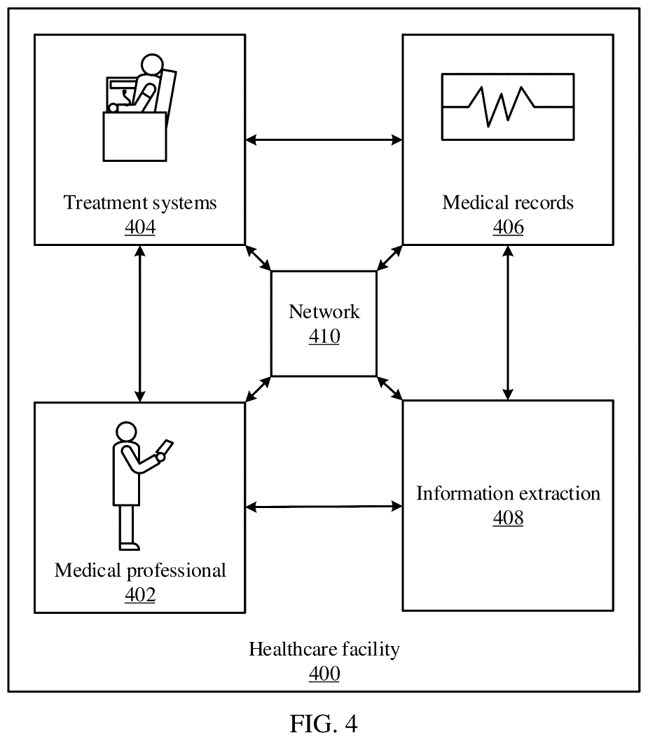

Referring now to FIG. 4, a diagram of information extraction is shown in the context of a healthcare facility 400. Information extraction 408 may be used to process patient information, for example by identifying relevant facts from patient history information and medical records 406. The information extraction 408 may be implemented with a large language model using a set of domain-specific training examples that are labeled in accordance with their usage in a medical context. A large amount of patient information can be rapidly processed in this manner to identify important information, such as exposure to allergens, medications taken, and the circumstances of an injury.

The healthcare facility may include one or more medical professionals 402 who review information extracted from a patient's medical records 406 to determine their healthcare and treatment needs. These medical records 406 may include self-reported information from the patient, test results, and notes by healthcare personnel made to the patient's file. Treatment systems 404 may furthermore monitor patient status to generate medical records 406 and may be designed to automatically administer and adjust treatments as needed.

Based on information drawn from the information extraction 408, the medical professionals 402 may then make medical decisions about patient healthcare suited to the patient's needs. For example, the medical professionals 402 may make a diagnosis of the patient's health condition and may prescribe particular medications, surgeries, and/or therapies.

The different elements of the healthcare facility 400 may communicate with one another via a network 410, for example using any appropriate wired or wireless communications protocol and medium. Thus information extraction 408 receives textual information from medical professionals 402, from treatment systems 404, from medical records 406, and updates the medical records 406 with the output of the language model, and may coordinate with treatment systems 404 in some cases to automatically administer or alter a treatment. For example, if the information extraction 408 indicates a potential allergic reaction to a treatment or medication, the treatment systems 404 may automatically halt the administration of the treatment.

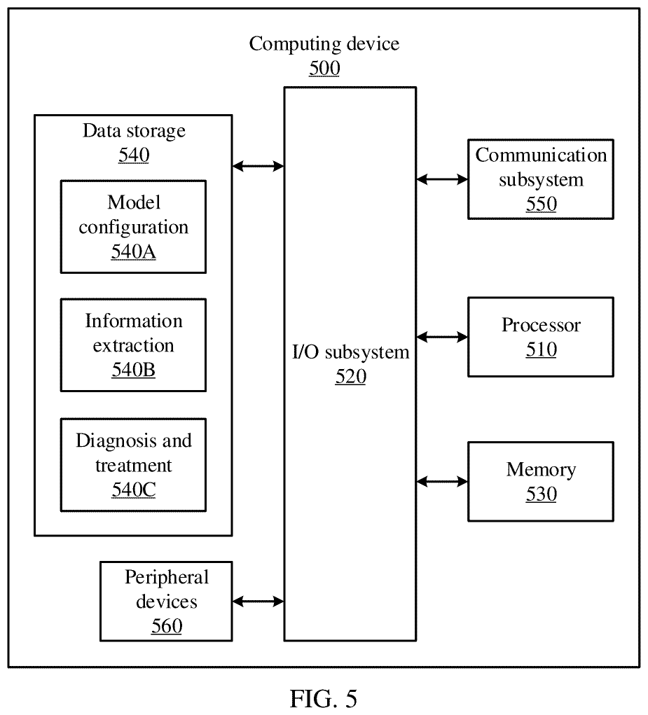

Referring now to FIG. 5, an exemplary computing device 500 is shown, in accordance with an embodiment of the present invention. The computing device 500 is configured to perform tissue analysis.

The computing device 500 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 500 may be embodied as one or more compute sleds, memory sleds, or other racks, sleds, computing chassis, or other components of a physically disaggregated computing device.

As shown in FIG. 5, the computing device 500 illustratively includes the processor 510, an input/output subsystem 520, a memory 530, a data storage device 540, and a communication subsystem 550, and/or other components and devices commonly found in a server or similar computing device. The computing device 500 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 530, or portions thereof, may be incorporated in the processor 510 in some embodiments.

The processor 510 may be embodied as any type of processor capable of performing the functions described herein. The processor 510 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 530 may be embodied as any type of volatile or non-volatile memory or data storage capable of performing the functions described herein. In operation, the memory 530 may store various data and software used during operation of the computing device 500, such as operating systems, applications, programs, libraries, and drivers. The memory 530 is communicatively coupled to the processor 510 via the I/O subsystem 520, which may be embodied as circuitry and/or components to facilitate input/output operations with the processor 510, the memory 530, and other components of the computing device 500. For example, the I/O subsystem 520 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 520 may form a portion of a system-on-a-chip (SOC) and be incorporated, along with the processor 510, the memory 530, and other components of the computing device 500, on a single integrated circuit chip.

The data storage device 540 may be embodied as any type of device or devices configured for short-term or long-term storage of data such as, for example, memory devices and circuits, memory cards, hard disk drives, solid state drives, or other data storage devices. The data storage device 540 can store program code 540A for configuring a model, 540B for performing information extraction, and/or 540C for performing diagnosis and treatment. Any or all of these program code blocks may be included in a given computing system. The communication subsystem 550 of the computing device 500 may be embodied as any network interface controller or other communication circuit, device, or collection thereof, capable of enabling communications between the computing device 500 and other remote devices over a network. The communication subsystem 550 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 500 may also include one or more peripheral devices 560. The peripheral devices 560 may include any number of additional input/output devices, interface devices, and/or other peripheral devices. For example, in some embodiments, the peripheral devices 560 may include a display, touch screen, graphics circuitry, keyboard, mouse, speaker system, microphone, network interface, and/or other input/output devices, interface devices, and/or peripheral devices.

Of course, the computing device 500 may also include other elements (not shown), as readily contemplated by one of skill in the art, as well as omit certain elements. For example, various other sensors, input devices, and/or output devices can be included in computing device 500, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 500 are readily contemplated by one of ordinary skill in the art given the teachings of the present invention provided herein.

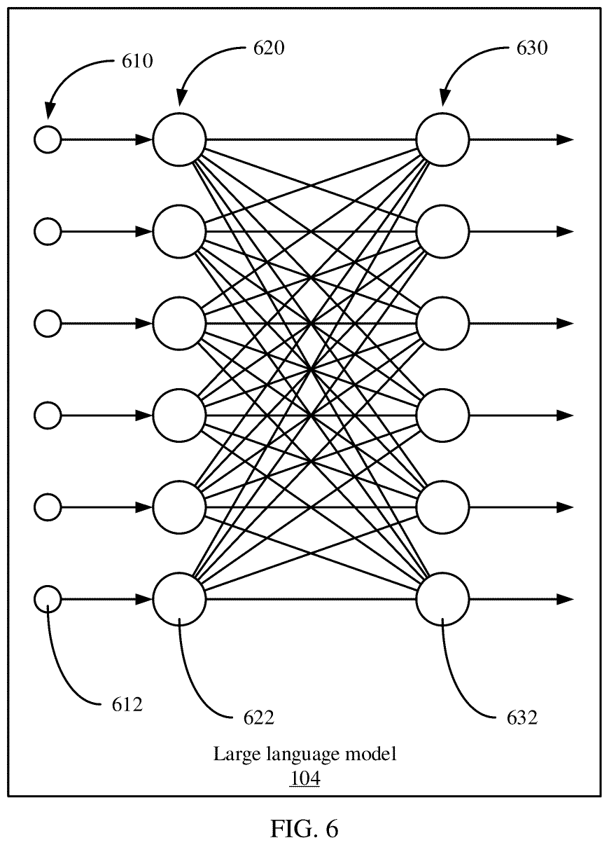

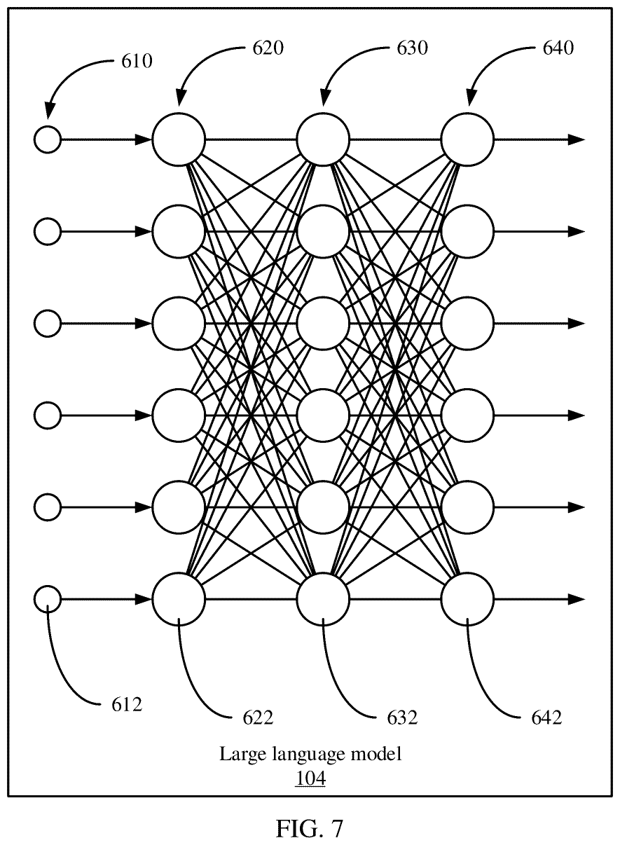

Referring now to FIGS. 6 and 7, exemplary neural network architectures are shown, which may be used to implement parts of the present models, such as the large language model 104. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values.

The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 620 of source nodes 622, and a single computation layer 630 having one or more computation nodes 632 that also act as output nodes, where there is a single computation node 632 for each possible category into which the input example could be classified. An input layer 620 can have a number of source nodes 622 equal to the number of data values 612 in the input data 610. The data values 612 in the input data 610 can be represented as a column vector. Each computation node 632 in the computation layer 630 generates a linear combination of weighted values from the input data 610 fed into input nodes 620, and applies a non-linear activation function that is differentiable to the sum. The exemplary simple neural network can perform classification on linearly separable examples (e.g., patterns).

A deep neural network, such as a multilayer perceptron, can have an input layer 620 of source nodes 622, one or more computation layer(s) 630 having one or more computation nodes 632, and an output layer 640, where there is a single output node 642 for each possible category into which the input example could be classified. An input layer 620 can have a number of source nodes 622 equal to the number of data values 612 in the input data 610. The computation nodes 632 in the computation layer(s) 630 can also be referred to as hidden layers, because they are between the source nodes 622 and output node(s) 642 and are not directly observed. Each node 632, 642 in a computation layer generates a linear combination of weighted values from the values output from the nodes in a previous layer, and applies a non-linear activation function that is differentiable over the range of the linear combination. The weights applied to the value from each previous node can be denoted, for example, by w₁, W₂, . . . . Wₙ₋₁, Wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 632 in the one or more computation (hidden) layer(s) 630 perform a nonlinear transformation on the input data 612 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor-or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for information extraction, comprising:
configuring a language model with an information extraction instruction prompt and at least one labeled example prompt;
validating configuration of the language model using at least one validation prompt;
correcting errors made by the language model in response to the at least one validation prompt using a correction prompt;
performing information extraction on an unlabeled sentence using the language model to identify a relation from the unlabeled sentence; and
performing an action responsive to the identified relation.

2. The method of claim 1, wherein the information extraction instruction prompt and the at least one labeled example prompt include a textual description of an information extraction task, including a definition of a relation format.

3. The method of claim 1, wherein the at least one labeled example prompt is drawn from a set of training data that includes sentences and associated relations.

4. The method of claim 3, wherein the at least one validation prompt is also drawn from the set of training data.

5. The method of claim 4, wherein the correction prompt identifies a response to the at least validation prompt that does not match a label of the at least one validation prompt from the training data and provides supplies the language model with the label.

6. The method of claim 1, wherein the at least one labeled example prompt includes a confidence score and wherein inputting the test prompt to the language model further determines a confidence score associated with the relation.

7. The method of claim 1, wherein the unlabeled sentence relates to a patient's medical condition.

8. The method of claim 7, wherein performing the action includes automatically adjusting a patient's treatment based on the identified relation.

9. The method of claim 7, wherein the identified relation is stored in a medical history of the patient to aid in medical decision making by a healthcare professional.

10. The method of claim 1, wherein the language model is a pretrained large language model based on a machine learning model.

11. A system for information extraction, comprising:
a hardware processor; and
a memory that stores a computer program which, when executed by the hardware processor, causes the hardware processor to:
configure a language model with an information extraction instruction prompt and at least one labeled example prompt;
validate configuration of the language model using at least one validation prompt;
correct errors made by the language model in response to the at least one validation prompt using a correction prompt;
perform information extraction on an unlabeled sentence using the language model to identify a relation from the unlabeled sentence; and
perform an action responsive to the identified relation.

12. The system of claim 11, wherein the information extraction instruction prompt and the at least one labeled example prompt include a textual description of an information extraction task, including a definition of a relation format.

13. The system of claim 11, wherein the at least one labeled example prompt is drawn from a set of training data that includes sentences and associated relations.

14. The system of claim 13, wherein the at least one validation prompt is also drawn from the set of training data.

15. The system of claim 14, wherein the correction prompt identifies a response to the at least validation prompt that does not match a label of the at least one validation prompt from the training data and provides supplies the language model with the label.

16. The system of claim 11, wherein the at least one labeled example prompt includes a confidence score and wherein inputting the test prompt to the language model further determines a confidence score associated with the relation.

17. The system of claim 11, wherein the unlabeled sentence relates to a patient's medical condition.

18. The system of claim 17, wherein the computer program further causes the hardware processor to automatically adjust a patient's treatment based on the identified relation.

19. The system of claim 17, wherein the identified relation is stored in a medical history of the patient to aid in medical decision making by a healthcare professional.

20. The system of claim 11, wherein the language model is a pretrained large language model based on a machine learning model.
